(19) United States
(12) Patent Application Publication
(10) Pub. No.: US 2020/0016187 A1
(43) Pub. Date: Jan. 16, 2020

(54) MODIFICATION OF THE PH AND OTHER PHYSICAL PROPERTIES OF NICOTINAMIDE ADENINE DINUCLEOTIDE (OXIDIZED FORM) TO ALLOW FOR ENHANCED STABILITY AND MULTIPLE DELIVERY SYSTEMS

(71) Applicant: Robert Michael VASIL, II, Carlsbad, CA (US)

(72) Inventor: Robert Michael VASIL, II, Carlsbad, CA (US)

(21) Appl. No.: 16/285,875

(22) Filed: Feb. 26, 2019

Related U.S. Application Data
(60) Provisional application No. 62/697,204, filed on Jul. 12, 2018.
(60) Provisional application No. 62/789,086, filed on Jan. 7, 2019.

(51) Int. Cl. A61K 31/7084; A61K 9/00; A61K 47/26; A61K 47/02; A61K 47/38; A61K 47/22

(52) U.S. Cl. CPC A61K 31/7084; A61K 9/0056; A61K 47/22; A61K 47/02; A61K 47/38; A61K 47/26

(57) ABSTRACT

The present disclosure relates to nicotinamide adenine dinucleotide (oxidized form) preparations and the use of hygroscopic components to prevent decomposition of the nicotinamide adenine dinucleotide (oxidized form). These compounds can then be prepared and administered nasally, sublingually, vaginally, rectally and topically to the skin to serve as nutritional supplements, medical foods or drugs. These compounds can also be used in the manufacture of nicotinamide adenine dinucleotide (oxidized form) preparations in transdermal patches, and inhalation preparations.

This application claims benefit to U.S. provisional application No. 62/697,204, filed Jul. 12, 2018; and U.S. provisional application No. 62/789,086, filed Jan. 7, 2019, the contents of each of which is incorporated by reference herein in their entirety.

### BACKGROUND

Unless otherwise noted, all documents referred to herein are incorporated by reference in their entirety.

The present disclosure relates to nicotinamide-adenine-dinucleotide in its oxidized form (“NAD+”) (see https://medicaldictionary.thefreedictionary.com/nicotinamide+adenine+dinucleotide) which occurs in all living cells, including human cells, and is a cofactor for enzymatic oxidation reactions. In metabolism, NAD+ is involved in redox reactions, carrying electrons from one reaction to another.

Lowry, O. H., et al. (Passonneau, J. V., & Rock, M. K. (1961 “The Stability of Pyridine Nucleotides,” The Journal of Biological Chemistry, 236(10) 2756-2859 (1961) notes that NAD+ is very hygroscopic and in an anhydrous state should be stored without exposure to water, otherwise known as a desiccated state. NAD+ dissolved in aqueous solutions between pH 2-6, stored at −70° C., can be stable for up to 6 months. Fairly neutral aqueous solutions are stable at 0° C. for up to 2 weeks. Solutions are rapidly degraded upon heating and are very labile in alkaline solutions. The rates of the decomposition of NAD+ in aqueous solutions are reported and are found to be quite rapid. In addition UV light has been shown to degrade NAD+. Lowry also concluded that aqueous solutions of NAD+ are very labile in the presence of phosphates, maleates, or carbonates.

Bioavailability studies performed by Baum, C. L. et al. (“The hydrolysis of nicotinamide adenine nucleotide by brush border membranes of rat intestine,” Biochemical Journal, 204(1), 203-207. doi:10.1042/bj2040203 (1982)) and Gross, C. J., et al. (“Digestion and Absorption of NAD by the Small Intestine of the Rat,” Journal of Nutrition, 113(2), 412-420 (1983)) indicated that ingested NAD+ was primarily hydrolyzed in the small intestine by brush border cells. The conclusions reached were that a pyrophosphatase present in the intestinal juice and to a much lesser extent in the pancreatic juice releases 5′-AMP and nicotinamide ribonucleotide. The 5′-AMP was rapidly converted to adenosine then to inosine by bacteria-free intestinal contents.

As described by Narang, N., et al. (“Sublingual Mucosa as a Route for Systemic Drug Delivery,” International Journal of Pharmacy and Pharmaceutical Sciences, 3(3), 8-22 (2001), retrieved from https://innovareacademics.in/journal/ijpps/Vol3Supp12/1092.pdf), the absorption of a nutritional supplement, medical food, or drug through the sublingual route is 3 to 10 times greater than oral route. In terms of permeability, the sublingual area of the oral cavity (i.e. the floor of the mouth) is more permeable than the buccal (cheek) area, which in turn is more permeable than the palatal (roof of the mouth) area. The portion of drug absorbed through the sublingual blood vessels bypasses the hepatic first-pass metabolic processes giving acceptable bioavailability.

As stated by Canto, C., et al. (“NAD(+) Metabolism and the Control of Energy Homeostasis: A Balancing Act between Mitochondria and the Nucleus,” Cell Metabolism, 22(1), 31-53 (2015)) NAD+ precursors are metabolized very quickly in mammalian blood and tissues. Plasma levels of most NAD+ precursors are unable to systematically sustain high levels of NAD+. NAD+ levels increase in response to energy stresses, such as glucose deprivation, (see Fulco, M., et al., “Glucose restriction inhibits skeletal myoblast differentiation by activating SIRT1 through AMPK-mediated regulation of Nampt,” Developmental Cell, 14(5), 661-73 (2008)), fasting, exercise (see Canto, C., et al, “Interdependence of AMPK and SIRT1 for metabolic adaptation to fasting and exercise in skeletal muscle,” Cell Metab., 11(3), 213-219 (2010), retrieved Feb. 2, 2019, from https://www.ncbi.nlm.nih.gov/pmc/articles/PMC3616265/), and caloric restriction (see Chen, D., et al., (2008), “Tissue-specific regulation of SIRT1 by calorie restriction.” Genes & Development, 22(13), 1753-7 (2008)).

NAD+ is currently being used in drug rehabilitation clinics to help combat withdrawal symptoms. The NAD+ used in this manner is administered intravenously. This delivery method is effective in delivering NAD+ to the bloodstream, with a properly prepared pH balanced solution, since it is delivered quickly, before the redox reaction degrades the NAD+ molecule and renders it ineffective. In order to be effective, this type of intravenous administration of NAD+ must be performed in a medical setting, thus increasing its costs and reducing its convenience. Therefore, it would be desirable if a different delivery system for stable NAD+ preparations were developed.

The present inventor found that NAD+ is an acidic compound which requires a pH buffer in order to prevent loss of tooth enamel and discomfort that would otherwise result from long term use of oral drops, sublingual lozenges, and buccal lozenges containing NAD+. The present inventor also found that a pH buffer is also needed in order to prevent discomfort in the anal and vaginal areas from anal and vaginal suppositories, discomfort in the lungs and nasal passages from inhaled preparations, and discomfort in the skin from topical applications. All of these delivery methods face a problem, given the acidity of NAD+. So it would be desirable to find a pH modifier and other physical modifiers of NAD+ that do not compromise the integrity of the molecule.

In addition, NAD+ has typically been administered intravenously as a nutritional supplement, medical food, or drug, and most of the stability research has been reported with NAD+ being in an aqueous solution. Due to the inconvenience of intravenous injections, the present inventor set out on a path to ascertain what ingredients would be stable when mixed with the anhydrous form of NAD+ for the production of sublingual lozenges, tablets, drops; nasal sprays or powder; inhalation preparations; topical lotions and creams; transdermal patches; and vaginal or rectal suppositories

### SUMMARY OF THE INVENTION

In accordance with the first aspect of the present disclosure, NAD+'s pH is buffered with the addition of other physical modifiers without inducing a redox reaction that would degrade NAD+ and the resulting composition may be administered:
Sublingually in the form of lozenges, or tablets administered underneath the tongue for absorption;
Sublingually in the form of drops administered underneath the tongue for absorption;
Intranasally as a liquid spray or a powder spray through the nostrils and into the nasal passages to be absorbed;
Rectally/Vaginally in the form of suppositories inserted up the rectum/vagina; or
Topically in the form of an oil based lotion or cream to be applied to the skin for absorption.

One embodiment of the present disclosure is a method of adjusting the pH of NAD+ without inducing a redox reaction by adding a hygroscopic compound.

Another embodiment of the present disclosure is the method described above, wherein said hygroscopic compound is a magnesium carbonate.

Another embodiment of the present disclosure is the method described above, wherein said hygroscopic compound has a white color.

Another embodiment of the present disclosure is a composition comprising NAD+ and a hygroscopic compound.

Another embodiment of the present disclosure is the composition described above, wherein said hygroscopic compound is magnesium carbonate.

Another embodiment of the present disclosure is a method of adjusting the taste of an NAD+ composition to reduce bitterness comprising a step of adding a hygroscopic sweetener to NAD+.

Another embodiment of the present disclosure is the method described above wherein said hygroscopic sweetener is stevia.

Another embodiment of the present disclosure is a composition comprising NAD+ and a hygroscopic sweetener compound.

Another embodiment of the present disclosure is the composition described above, wherein said hygroscopic sweetener is lactose.

Another embodiment of the present disclosure is a method of producing a compactable lozenge or tablet comprising NAD+ without inducing degradation of NAD+, said method comprising the addition of a hygroscopic binder agent to NAD+.

Another embodiment of the present disclosure is the method described above wherein said hygroscopic binder agent is lactose.

Another embodiment of the present disclosure is a method of producing a compactable lozenge or tablet comprising NAD+ without inducing degradation of NAD+, the method comprising the addition of a hygroscopic release agent to NAD+.

Another embodiment of the present disclosure is the method described above wherein said hygroscopic release agent is silicon dioxide.

Another embodiment of the present disclosure is a lozenge or tablet comprising NAD+ and a hygroscopic release agent.

Another embodiment of the present disclosure is a composition comprising NAD+ and one or more components selected from the group consisting of hygroscopic pH modifiers, hygroscopic taste modifiers, hygroscopic binding agents, and hygroscopic release agents, wherein the pH of the hygroscopic pH modifiers, hygroscopic taste modifiers, hygroscopic binding agents, and hygroscopic release agents is not less than or equal to 4 and not greater than or equal to 9, wherein the composition does not comprise a hygroscopic pH modifier, hygroscopic taste modifier, hygroscopic binding agent, or hygroscopic release agent that has a

Another embodiment of the present disclosure is the composition described above, in which the one or more components are selected from the group consisting of magnesium carbonate, lactose, stevia, and silicon dioxide.

Another embodiment of the present disclosure is the composition described above, further comprising a sealed container surrounding the NAD+ and the one or more components.

Another embodiment of the present disclosure is the composition described above, wherein said composition is used as a nutritional supplement, a medical food, or a drug.

19. Another embodiment of the present disclosure is the composition described above which does not contain any material having a pH less than or equal to 4 or greater than or equal to 9.

One embodiment of the present disclosure is a composition comprising NAD+ and a hygroscopic basic compound which does not provide for the redox reaction of the NAD+ and which maintains the pH of the composition to be between 4 and 9. In one aspect of this embodiment, the composition does not comprise a hygroscopic compound having a pH equal to or below 4 or equal to or above 9. In another aspect of this embodiment, the composition does not comprise any material having a pH less than or equal to 4 or greater than or equal to 9.

Another embodiment of the present disclosure is a composition as described above, wherein the hygroscopic basic compound is a pH modifier that maintains the pH of the composition to be between 6.5 and 7.5.

Another embodiment of the present disclosure is a composition as described above, wherein said pH modifier is selected from the group consisting of magnesium carbonate and (MgCO₃), and calcium phosphate dibasic (CaHPO₄).

Another embodiment of the present disclosure is a composition as described above, further comprising a taste modifier that does not provide for a redox reaction of the NAD+.

Another embodiment of the present disclosure is a composition as described above, wherein the taste modifier is selected from the group consisting of mannitol, lactose, sodium chloride, dextrose, stevia, erythritol, sucrose, and xylitol.

Another embodiment of the present disclosure is a composition as described above, further comprising a binding agent that does not provide for a redox reaction of the NAD+.

Another embodiment of the present disclosure is a composition as described above, wherein the binding agent is selected from the group consisting of microcrystalline cellulose, lactose, dextrose, sucrose, and mannitol.

Another embodiment of the present disclosure is a composition as described above, further comprising a releasing agent that does not provide for a redox reaction of the NAD+.

Another embodiment of the present disclosure is a composition as described above, wherein the releasing agent is selected from the group consisting of ascorbyl palmitate and silicon dioxide.

Another embodiment of the present disclosure is a composition as described above, further comprising a coating that does not contain water.

Another embodiment of the present disclosure is a composition as described above, wherein said composition is formed as a sublingual lozenge, tablet, or drop; nasal sprays or powder; topical lotion or cream; inhalation preparation; transdermal patch; or vaginal or rectal suppository.

Another embodiment of the present disclosure is a composition as described above, wherein said composition is white.

Another embodiment of the present disclosure is a method of determining whether the NAD+ in a composition described above has degraded, comprising the step of not using the composition if its color is not white.

Another embodiment of the present disclosure is a combination comprising a package and a composition discussed above, wherein over a period of 24 months the combination prevents the NAD+ in the composition from being exposed to water in an amount that would degrade the NAD+.

Another embodiment of the present disclosure is a method of administering NAD+ to a human in need thereof, comprising sublingually, intranasally, rectally, vaginally, or topically administering a composition described above.

Another embodiment of the present disclosure is a liquid composition comprising NAD+ and a liquid solvent that has a pH greater than or equal to 4 and less than or equal to 9, wherein said liquid solvent does not provide for a redox reaction of the NAD+. Said composition may contain less than 2% water by weight.

Another embodiment of the present disclosure is the liquid composition described above, wherein the solvent is selected from the group consisting of oils, alcohols, acids, tocotrienols, and sugar alcohols.

Another embodiment of the present disclosure is a liquid composition as described above, wherein the composition, including said oils, acids, tocotrienols, alcohols, and sugar alcohols, does not contain water in an amount that would degrade the NAD+.

Another embodiment of the present disclosure is a viscous composition as described above, wherein said liquid is selected from the group consisting of MCT oil, mineral oil, vegetable oil, sorbitol, tocopherol acetate, benzyl alcohol, polysorbate 80, and glycerin.

Another embodiment of the present disclosure is a composition as described above, wherein said composition does not contain water.

Another embodiment of the present disclosure is a method of administering NAD+ to a human in need thereof, comprising topically administering a composition described above to the skin of the human.

For the purposes of the present disclosure, a human in need of NAD+ is a human needing NAD+ for reasons known in the art.

Another embodiment of the present disclosure is a composition comprising NAD+ and a compound selected from the group consisting of magnesium hydroxide and ascorbic acid.

### DETAILED DESCRIPTION OF THE INVENTION

As stated in (THE MERCK INDEX: AN ENCYCLOPEDIA OF CHEMICALS, DRUGS, AND BIOLOGICALS (O'Neil, M. J., (Ed.), (14th ed.), N J: Merck. (2006)), preparations of aqueous solutions containing NAD+ are prepared by adding either sodium hydroxide or sodium bicarbonate as a buffer to adjust the pH of the solution to 7. Further, it is known from Lowry, O., et al. (“The Stability of Pyridine Nucleotides,” The Journal of Biological Chemistry, 236(10), 2756-2859 (1961)) that NAD+ dissolved in an aqueous solution is reduced within a few days and that water acts as a catalyst accelerating decomposition. Based on this, one would have assumed that combining the anhydrous sodium hydroxide or sodium bicarbonate with the anhydrous form of NAD+ in a matrix would be an effective way to buffer the pH without degrading the NAD+. However, the present inventor found that this theory was wrong. The NAD+ when combined with sodium hydroxide or sodium bicarbonate and compressed into an anhydrous matrix showed degradation shortly after creation of the lozenge tablet. See Example 1. Even though it would seem simple to adjust the pH of NAD+ to produce sublingual lozenges, tablets, or drops; nasal sprays or powder; inhalation preparations; topical lotions and creams; transdermal patches; and vaginal or rectal suppositories, the present inventor found that adjusting the pH was an unexpected difficulty.

As described in Lowry, O. H., et al. (“The Stability of Pyridine Nucleotides. The Journal of Biological Chemistry,” 236(10), 2756-2859 (1961)), water acts as a catalyst for NAD+ and, when dissolved in neutral or slightly acidic solutions stored at zero degrees Fahrenheit, the stability of NAD+ will last up to 2 weeks. It is also known that the anhydrous form of NAD+ is hygroscopic and should be stored in a desiccated state, without exposure to ambient humidity.

Conversely, as described by Lowry, NAD+ dissolved in an aqueous solution is very labile in the presence of phosphates, maleates, or carbonates. Contrary to what would have been expected given Lowry's disclosure, and to the present inventor's surprise, when testing hygroscopic ingredients and their reactions with the powder form NAD+, it was found that the anhydrous forms of both magnesium carbonate and calcium phosphate dibasic, when combined as a matrix, caused little to no degradation of the NAD+.

One step in the manufacture of products such as sublingual lozenges, tablets, or drops; nasal sprays or powder; topical lotions and creams; inhalation preparations; transdermal patches; and vaginal or rectal suppositories using the anhydrous form of NAD+ may include the modification of the pH using one or more pH buffers that will not degrade the NAD+. This may be done by adding one or more pH buffers to the NAD+. In some embodiments of the present disclosure, the addition of one or more pH buffers to the NAD+ is the first step in the process of manufacturing, for example, sublingual lozenges, tablets, or drops; nasal sprays or powder; topical lotions and creams; inhalation preparations; transdermal patches; and vaginal or rectal suppositories.

The amount of NAD+ that may be present in certain embodiments of the present disclosure is not particularly limited so long as it is an amount sufficient to provide the effects sought. In some embodiments, the composition may contain up to 50% by weight NAD+. In other embodiments, the composition may contain up to 25%, up to 10%, up to 5%, or up to 1% by weight NAD+.

The present inventor found that some commonly used pH modifiers are not suitable for use with NAD+ without the addition of other materials because those pH modifiers may cause the NAD+ to degrade. For example, dicalcium phosphate dihydrate, potassium hydroxide, sodium hydroxide, potassium bicarbonate, sodium bicarbonate, calcium carbonate, magnesium oxide, and were found by the present inventor to cause degradation of the NAD+ when they were formulated, by themselves, with NAD+, as contrasted with a control. See the Examples below.

The present inventor unexpectedly found that only certain pH modifiers do not degrade NAD+ when formulated, by themselves, with NAD+. Suitable pH modifiers that may be used include, but are not limited to magnesium hydroxide, magnesium carbonate and calcium phosphate dibasic (CaHPO₄). For purposes of the present application, the term “pH modifier” encompasses materials that are pH modifiers and those that are pH buffers. Accordingly, these are preferred pH modifiers of the present disclosure.

The amount of pH modifier that may be present in the compositions of the present disclosure is an amount that provides for a pH at which the NAD+ does not degrade. If the pH of composition is too basic or too acidic (for example, equal to or above 9 or equal to or below 4), the NAD+ will degrade. In addition, if the pH of the composition is too basic or too acidic it will cause irritation or other discomfort when it is administered. Thus, it is preferred that the pH of the composition be below 9 and above 4. In an exemplary embodiment, the pH of the composition may be fairly neutral, for example from about 6.5 to about 7.5, preferably from 6.5 to 7.5, more preferably about 7, and most preferably 7. The amount of pH modifier that may be present in the composition may be, for example, any amount necessary to provide for the desired pH.

One aspect of the present disclosure is thus a method of adjusting the pH of NAD+ without inducing a redox reaction. This may be done in some preferred embodiments by adding magnesium carbonate, which is a hygroscopic basic compound, to NAD+. This method may also be performed by the selection of specific materials that have a pH in the ranges noted above.

Similarly, another aspect of the present disclosure is a composition comprising NAD+ and not including a material that has a pH at or above 9 or at or below 4. A preferred aspect of the present disclosure is a composition that comprises NAD+ and only materials that have a pH that is below 9 and above 4, preferably a pH of from about 6.5 to about 7.5, or a pH 6.5 to 7.5, or a pH of about 7, or a pH of 7. Such compositions may include materials that do not have a pH.

The present inventor found that the pH relationship noted above provided for stability in the NAD+, but surprisingly, magnesium hydroxide and ascorbic acid, which have pHs outside of the range of 4-9 surprisingly also provided for stable compositions of NAD+, whereas other materials having pHs outside of the range of 4-9 degraded the NAD+.

It may also be desirable to add one or more taste modifiers to the compositions of the present disclosure. Thus, another step in the manufacture of products such as sublingual lozenges, tablets, or drops; nasal sprays or powder; topical lotions and creams; inhalation preparations; transdermal patches; and vaginal or rectal suppositories using the anhydrous form of NAD+ may include the modification of the product's flavor profile via the addition of one or more taste modifier. In some embodiments, this step may occur after the addition of the pH modifier to the composition. Indeed, in some embodiments, the addition of a pH modifier may necessitate the addition of one or more taste modifiers, since some pH modifiers may provide for a poor tasting product, which is something that has not been previously reported. However, the present inventor found that adding taste modifiers was not a simple task, as the present inventor found that commonly used taste modifiers may degrade the NAD+.

The present inventor found that some commonly used taste modifiers are not suitable for use with NAD+ without the addition of other materials because those taste modifiers may cause the NAD+ to degrade. For example, citric acid was found by the present inventor to cause degradation of the NAD+ when it was formulated, by itself, with NAD+. See the Examples below.

The present inventor unexpectedly found that only certain taste modifiers do not degrade NAD+ when formulated, by themselves, with NAD+. Suitable taste modifiers that may be used include, but are not limited to sodium ascorbate, ascorbic acid, mannitol, lactose, sodium chloride, dextrose, stevia, erythritol, sucrose, and xylitol. Thus, in some embodiments, mannitol, lactose, sodium chloride, dextrose, stevia, erythritol, sucrose, and xylitol are preferred taste modifiers.

The amount of taste modifier that may be present in the compositions of the present disclosure is not particularly limited, and may be any amount necessary to achieve the desired taste modification.

Another step in the manufacture of products such as sublingual lozenges, tablets, or drops; nasal sprays or powder; topical lotions and creams; inhalation preparations; transdermal patches; and vaginal or rectal suppositories using the anhydrous form of NAD+ may include the addition of one or more binding agents. The one or more binding agents may be added to compensate for NAD+'s frailty and its tendency to crumble upon compression. In some embodiments, the one or more binding agents are added after the taste modifiers and pH modifiers are added to the composition. However, the present inventor discovered that the addition of one or more binding agents, or binders, could cause problems with the composition, namely the degradation of the NAD+.

The present inventor found that some commonly used binders are not suitable for use with NAD+ without the addition of other materials because those binders may cause the NAD+ to degrade. For example, polyvinylpyrrolidone was found by the present inventor to cause degradation of the NAD+ when it was formulated, by itself, with NAD+. See the Examples below.

The present inventor unexpectedly found that only certain binders do not degrade NAD+ when formulated, by themselves, with NAD+. Suitable binders that may be used include, but are not limited to microcrystalline cellulose, dextrose, mannitol, lactose, and sucrose. Thus, in some embodiments, microcrystalline cellulose, dextrose, mannitol, lactose, and sucrose are preferred binders.

The amount of binder that may be present in the compositions of the present disclosure is not particularly limited, and may be any amount that provides for the desired binding.

Another step in the manufacture of products such as sublingual lozenges, tablets, or drops; nasal sprays or powder; topical lotions and creams; inhalation preparations; transdermal patches; and vaginal or rectal suppositories using the anhydrous form of NAD+ may include the addition of one or more releasing agents to the composition. The releasing agents may be added to allow for the products to be released from the mold. In some embodiments, the one or more releasing agents are added to the composition after the pH modifiers, taste modifiers, and binders. However, the present inventor discovered that the addition of one or more releasing agents could cause problems with the composition, namely the rapid degradation of the NAD+.

The present inventor found that some commonly used releasing agents are not suitable for use with NAD+ without the addition of other materials because those releasing agents may cause the NAD+ to degrade. For example, stearic acid and magnesium stearate were found by the present inventor to cause degradation of the NAD+ when they were formulated, by themselves, with NAD+. See the Examples below.

The present inventor unexpectedly found that only certain releasing agents do not degrade NAD+ when formulated, by themselves, with NAD+. Suitable releasing agents that may be used include, but are not limited to ascorbyl palmitate, and silicon dioxide. Thus, in some embodiments, ascorbyl palmitate, and silicon dioxide are preferred releasing agents.

The amount of releasing agent that may be present in the compositions of the present disclosure is not particularly limited, and may be any amount that provides for the desired releasing effect.

Another step in the manufacture of products such as sublingual lozenges, tablets, or drops; nasal sprays or powder; topical lotions and creams; inhalation preparations; transdermal patches; and vaginal or rectal suppositories using the anhydrous form of NAD+ may include the addition of one or more bulking agents or delivery modifiers. However, the present inventor found that certain bulking agents and delivery modifiers, such as corn starch and gelatin capsules, respectively, caused the degradation of the NAD+ when they were formulated, by themselves, with NAD+. See the Examples below.

As examples, the following materials, when mixed with the anhydrous form of NAD+, where found to have degraded the NAD+ molecule after a two week period (see the Examples below):

Potassium Hydroxide, KOH (pH modifier and buffer)

Sodium hydroxide, NaOH (pH modifier and buffer)

Potassium bicarbonate, K₂CO₂ (pH modifier and buffer)

Sodium bicarbonate Na₂CO₂ (pH modifier and buffer)

Magnesium oxide, MgO (pH modifier and buffer)

Corn starch (bulking agent)

Stearic acid (releasing agent)

Citric acid (taste modifier)

Calcium carbonate, CaCO₂ (pH modifier and buffer)

Dicalcium phosphate dihydrate (pH modifier and binder)

Magnesium stearate (releasing agent)

Polyvinylpyrolidone (binder)

Gelatin capsules (delivery modifier)

As discussed above, the materials listed above are common in the manufacturing of sublingual lozenges, tablets, or drops; nasal sprays or powder; topical lotions and creams; inhalation preparations; transdermal patches; and vaginal or rectal suppositories. However, as a result of their hygroscopic and highly basic or acidic properties, the present inventor found that they degraded the NAD+ molecule, thereby presenting a problem with the manufacturing and use of compositions of NAD+.

The following materials, when mixed with the anhydrous form of NAD+, were found to not degrade the NAD+ molecule after a two week period:

Rice flour (bulking agent)

Lactose (taste modifier, binding agent)

Sodium chloride, NaCl (taste modifier)

Microcrystalline cellulose (binding agent)

Magnesium carbonate, MgCO₂ (pH modifier)

Dextrose (taste modifier, binding agent)

Ascorbyl palmitate (release agent)

Stevia (taste modifier)

Erythitol (taste modifier)

Sucrose (taste modifier, binding agent)

Mannitol (taste modifier, binding agent)

Xylitol (taste modifier)

Silicon dioxide (release agent)

Sodium ascorbate (taste modifier)

Ascorbic acid (taste modifier)

The present disclosure also relates to liquid compositions containing NAD+. The present inventor unexpectedly found that, when mixed with or dissolved in certain aqueous and nonaqueous solutions or solvents, NAD+ did not show much, if any, degradation. The present inventor found that NAD+ degraded in aqueous and nonaqueous solutions or solvents when it was introduced to materials having either high or low acidity levels. Thus, the present inventor found both that it is possible to prepare stable, aqueous and nonaqueous solutions or solvent compositions of NAD+ using aqueous and nonaqueous solutions or solvent products having fairly neutral pH or no pH, and also that it is possible to select suitable aqueous and nonaqueous solutions or solvents components with which to combine NAD+ based upon their pH neutrality and low water content.

When mixed with the anhydrous form of NAD+ after a one week period, the aqueous and nonaqueous solutions or solvent products below were found to not rapidly degrade the NAD+ molecule:

Sorbitol

Benzyl alcohol

Tocopherol acetates

Polysorbate 80

Glycerin

MCT oil

Mineral oil

Vegetable oil

Accordingly, in certain embodiments of the present disclosure, aqueous and nonaqueous solutions or solvent products that may be used with NAD+ include oils, viscous solvents, and solutions that contain minimal to no water content, so as to not degrade the NAD+. Solvents that have no pH or which have a pH of more than 4 and less than 9 may also be used in certain embodiments. Solvents having the pHs previously discussed herein may be used in other preferred embodiments. In preferred embodiments, oils, viscous solvents, and solutions that contain small amounts of water with no pH or neutral pH levels more than 4 and less than 9 may be used.

One embodiment of the present disclosure is a topical formulation, which may be in the form of a cream or lotion. In this embodiment, the pH of NAD+ may be buffered with the addition of other physical modifiers, without inducing the redox reaction and may be administered topically. Other materials as discussed previously may be used in this embodiment, as well. An example of a topical lotion formulation includes 6% by weight NAD+, 6% by weight calcium phosphate dibasic (as a pH buffer), 3% by weight tocopherol (Vitamin E), 20% by weight glycerin, and 65% by weight MCT oil.

The compositions of the present disclosure should be free from amounts of water that would provide for the degradation of NAD+. The maximum amount of water that may be present is based on the specific additives that are present. For example, some compositions of the present disclosure may be nonaqueous compositions that contain less than 2% water, less than 1% water, less than 0.5% water, or may be anhydrous. Other compositions may contain a higher water content so long as the hygroscopic compound is capable of preventing the degradation of the NAD+. Typically, increased water content may provide for more rapid degradation of the NAD+.

The compositions of the present disclosure should also not be exposed to high temperatures, in order to maintain the stability of the NAD+. In preferred embodiments, the compositions are not exposed to temperatures above 85° F., more preferably the compositions are not exposed to temperatures above 75° F., or above room temperature, which for the purpose of this application is considered to be 68° F. Compositions according to the present disclosure may be stored at refrigerated temperatures, for example temperatures ranging from 32° F. or lower to room temperature. Typically, increased temperature may provide for more rapid degradation of the NAD+.

Another embodiment of the present disclosure is a spray formulation including NAD+. An example of a spray formulation according to this embodiment includes 300 mg NAD+, 300 mg calcium phosphate dibasic (used as a PH buffer), and 15 ml sorbitol. The manner of making and content of the spray formulations are not particularly limited, and spray formulations may be made according to methods known in the art.

Another embodiment of the present disclosure is a formulation for intranasal administration. In a formulation for intranasal administration, the NAD+ may be present in any form that allows for the effective administration of NAD+ into the nasal passages, including but not limited to nose drops, liquid sprays, or powder sprays. The manner of making and content of the intranasal formulations are not particularly limited, and intranasal formulations may be made according to methods known in the art.

Another embodiment of the present disclosure is a formulation providing for sublingual administration of NAD+. An example of a sublingual drop according to this embodiment includes 300 mg NAD+, 300 mg calcium phosphate dibasic (used as a PH buffer), 2 ml glycerin, and 8 ml sorbitol. The manner of making and content of the sublingual formulations are not particularly limited, and sublingual formulations may be made according to methods known in the art.

In order to provide for administration of sublingual drops, it is preferable the pH of the sublingual drop not be below 7 and that the sublingual drop contains a low water content. It is also preferable that the sublingual drop not be exposed to high heat. Such conditions will help in ensuring the stability of the NAD+, and may also help prevent the loss of tooth enamel from long term use, while also helping to prevent discomfort from the naturally acidic nature of NAD+.

Another embodiment of the present disclosure is a sublingual lozenge providing for sublingual administration of NAD+. An example of a sublingual lozenge tablet according to this embodiment includes 60.8 mg NAD+, 60.2 mg magnesium carbonate (used as a PH buffer), 464.5 mg lactose (used as a binding agent and taste modifier), 8 mg stevia (used as a taste modifier), and 8 mg silicon dioxide (used as a releasing agent).

In order to provide for the administration of sublingual lozenge tablets, it is preferable the pH of the sublingual drop not be below 7 and that the sublingual drop contains a low water content. It is also preferable that the sublingual drop not be exposed to high heat. Such conditions will help in ensuring the stability of the NAD+, and may also help prevent the loss of tooth enamel from long term use, while also helping to prevent discomfort from the naturally acidic nature of NAD+.

Another aspect of the present disclosure is providing a composition that permits a consumer to easily understand that the composition is stable. Doing so can be challenging. However, certain embodiments of the present disclosure are compositions that include white materials, such that the compositions are white in color. Degraded compositions may appear to have a yellow tint. Degradation may occur, for example, after a container has been opened, when water vapor, UV light, or humidity may degrade the product. Degradation may also occur if a container does not adequately shield the composition from conditions that degrade it. A white composition will permit a consumer to quickly determine whether the composition has degraded. Thus, a consumer may be confident that a white-colored composition, in this embodiment, is a non-degraded composition, whereas a composition that has, for example, a yellow tint may have suffered some degradation. It is possible that in other embodiments the composition may be altered such that colors other than white and yellow would be indicative of the state of the composition.

Being able to determine whether a composition of the present disclosure has degraded, or exceeded its shelf life, is important to consumers. The shelf life for such compositions may be, for example, up to 24 months, or even longer. Such expiration dates are usually stamped or adhered to the container in which the composition is located. A visual inspection of the composition, confirming that it is white and has not changed to, for example, a yellow tint, will permit consumers to understand that the composition has not degraded, despite perhaps having been in a container for close to, or even more than the shelf life of the product, for example, 24 months. Accordingly, a consumer may choose to not use a composition that is off-color.

Some embodiments of the present disclosure include a composition comprising NAD+ as described herein, surrounded by a container. The container is not particularly limited, but is preferably one that is water tight, desiccated, and provides shielding from UV light.

The container may be packaging or may be a coating or an encapsulating material, in certain embodiments. Preferably the container, packaging, coating, or encapsulating material does not contain an amount of water that would provide for the degradation of the NAD+. Preferably, the container, packaging, coating, or encapsulating material does not contain water. The container, packaging, coating, or encapsulating material preferably prevents the ingress of water or UV light, in amounts that would degrade the NAD+, over the course of the shelf life of the composition containing NAD+, which may be, for example, 24 months. In other preferred embodiments, the container, packaging, coating, or encapsulating material includes a desiccant. An example of an encapsulating material that may provide for the degradation of the NAD+ is a gelatin capsule. Without wishing to be bound by theory, the present inventor believes that the water content present in the gelatin capsule was itself enough to provide for the degradation of the NAD+. As stated by Biyani, M. K. et al. (Choosing Capsules: A Primer, Pharmaceutical Technology, 41(10), 36-41 (Oct. 2, 2017)), the shell of hard gelatin capsules may contain 13-16% water content. See Table 1.

Certain embodiments of the present disclosure also include those compositions that exclude the presence of items that cause the degradation of NAD+. Some preferred embodiments of the present disclosure thus include compositions that exclude, either individually or in the aggregate, the materials found to degrade NAD+, such as those described herein.

### EXAMPLES

The present disclosure is further discussed below with reference to specific Examples. However, the present disclosure is not limited thereto.

### Example 1

Preparations of nicotinamide adenine dinucleotide (oxidized form) were compounded. A compressed lozenge was created including 100 mg nicotinamide adenine dinucleotide (oxidized form) and 100 mg of a test substance. The powders were compressed in a tablet mold and released. Each test tablet was then placed in a white 75cc HDPE container that was sealed and removed from UV light for two weeks at room temperature. Test substances are shown in Tables 1 and 2 and include:

Calcium phosphate dibasic, CaHPO₄.2H₂O (pH Modifier and buffer)

Potassium bicarbonate, K₂CO₂ (pH Modifier and buffer)

Sodium bicarbonate, Na₂CO₂ (pH Modifier and buffer)

Stevia (taste modifier)

Xylitol (taste modifier)

Sucrose (taste modifier)

Erythritol (taste modifier)

Dextrose (taste modifier)

Magnesium hydroxide, Mg(OH)₂ (pH modifier and buffer)

Rice flour (bulking agent)

Corn starch (bulking agent)

Silicon dioxide (releasing agent)

Stearic acid (releasing agent)

Citric acid (taste modifier)

Sodium chloride, NaCl (taste modifier)

Mannitol (taste modifier and lozenge binder)

Calcium carbonate, CaCO₂ (pH Modifier and buffer)

Dicalcium phosphate dihydrate (pH modifier and lozenge binder)

Magnesium oxide, MgO (pH Modifier and buffer)

Magnesium stearate (releasing agent)

Ascorbyl palmitate (releasing agent)

Microcrystalline cellulose (bulking agent)

Lactose (taste modifier and lozenge binder)

Magnesium carbonate (MgCO₂) (pH modifier and buffer)

Ascorbic acid (taste modifier)

Sodium ascorbate (taste modifier)

Polyvinylpyrrolidone (binder)

Gelatin capsules

After a two week period, the compositions were tested by performing an HPLC UV. Compounds that were found to be effective additives to nicotinamide adenine dinucleotide (oxidized form) preparations were ones that minimized the amount of degradation present and which did not cause rapid decomposition of nicotinamide adenine dinucleotide (oxidized form) as compared to a control which included only NAD+. These results are shown in Tables 1 and 2. For example, the compositions in which the recovery (%) was above the recovery of the control were found to be effective additives. Table 1 reflects the 4% recovery margin, which is standard for such measurements. Accordingly, mannitol, Mg(OH)₂, and CaHPO₄ were deemed effective for the purposes of this test.

TABLE 1

| | | | Sample | | |
|---|---|---|---|---|---|
| | | | concen- | | |
| | Weight | Dilution | tration | Potency | Recovery |
| Test substance | (mg) | (ml) | (mg/ml) | (%) | (%) |
| Comparative | 26 | 5 | 5.2 | 3.333 | 65.8 |
| Example 1 | | | | | |
| Magnesium Oxide | 3 | 1 | 3 | 50 | 71.6 |
| MgO | | | | | |
| Citric acid | 5.5 | 3 | 1.83 | 50 | 74.6 |
| Dicalcium Phosphate | 1.7 | 1 | 1.7 | 50 | 79 |
| Dihydrate | | | | | |
| CaHPO₄ | | | | | |
| Stearic acid | 2.7 | 1 | 2.7 | 50 | 82.9 |
| Calcium Carbonate | 1.1 | 1 | 1.1 | 50 | 85.8 |
| CaCO₂ | | | | | |
| Magnesium stearate | 2.6 | 1 | 2.6 | 50 | 87.6 |
| Corn starch | 6.2 | 3 | 2.07 | 50 | 88.2 |
| Sodium Bicarbonate | 5.4 | 5 | 1.08 | 50 | 91.2 |
| Na₂CO₂ | | | | | |
| Potassium | 9.5 | 3 | 3.17 | 50 | 92.3 |
| Bicarbonate | | | | | |
| K₂CO₂ | | | | | |
| Mannitol | 3.6 | 3 | 1.2 | 50 | 96.1 |
| Magnesium | 1.1 | 1 | 1.1 | 50 | 96.2 |
| Hydroxide | | | | | |
| Mg(OH)₂ | | | | | |
| Calcium Phosphate | 1.9 | 1 | 1.9 | 50 | 97.2 |
| Dibasic | | | | | |
| NAD+ pure (control) | 4.4 | 3 | 1.467 | 100 | 99.9 |
| Rice flour | 4.8 | 3 | 1.6 | 50 | 100.3 |
| Lactose | 3.4 | 3 | 1.13 | 50 | 100.3 |
| Sodium Chloride | 5.3 | 3 | 1.77 | 50 | 100.4 |
| NaCl | | | | | |
| Microcrystalline | 5.1 | 3 | 1.7 | 50 | 100.9 |
| cellulose | | | | | |
| Magnesium | 4 | 3 | 1.33 | 50 | 101 |
| carbonate MgCO₂ | | | | | |
| Dextrose | 3.9 | 3 | 1.3 | 50 | 101.2 |
| Ascorbyl palmitate | 5.6 | 3 | 1.87 | 50 | 101.5 |
| Stevia | 0.9 | 1 | 0.9 | 50 | 101.8 |
| Erythitol | 4.5 | 3 | 1.5 | 50 | 102.7 |
| Sucrose | 3.8 | 3 | 1.27 | 50 | 103.1 |
| Xylitol | 2.1 | 1 | 2.1 | 50 | 103.3 |
| Silicon dioxide | 2.5 | 1 | 2.5 | 50 | 103.8 |

Potassium hydroxide, KOH (a pH modifier and buffer) and sodium hydroxide, NaOH (a pH modifier and buffer) were also tested. However, it was not possible to obtain HPLC data on these compositions, since they bubbled, liquefied, turned yellow, and created heat when they were made, and thus HPLC data could not be obtained.

TABLE 2

| | Sample | | | Concen- | |
|---|---|---|---|---|---|
| | weight | Volume | Potency | tration | Recovery |
| Test substance | (mg) | (ml) | (%) | (mg/ml) | (%) |
| Ascorbic acid | 31.1 | 14 | 50 | 1.083 | 97.5 |
| Sodium ascorbate | 34.1 | 14 | 50 | 1.19 | 97.9 |
| Polyvinylpyrrolidone | 7.9 | 7 | 50 | 0.52 | 92.9 |
| Tocopherol acetates | 14.5 | 14 | 50 | 0.512 | 98.9 |
| Tocopherols | 12.1 | 14 | 50 | 0.425 | 98.3 |
| NAD+ pure | 4.3 | 5 | 100 | 0.851 | 98.95 |

### Example 2

A lozenge tablet was created using 60.8 mg anhydrous nicotinamide adenine dinucleotide (oxidized form), 60.2 mg magnesium carbonate, 464.5 mg lactose, 8 mg stevia, and 8 mg silicon dioxide. The tablet was compressed, then stored in a 50cc plastic container with a 2 gram desiccant pack. The container was sealed and opened after 1 month. Stability testing was performed and indicated no NAD+ decomposition, with a measured recovery of 100.12%. After testing, the lozenge tablet was inserted back into the same plastic container and sealed.

### Example 3

The plastic container from Example 2 was reopened after 2 months. Stability testing was performed and indicated little to no NAD+ decomposition, with a measured recovery of 99.18%.

### Example 4

Preparations of nicotinamide adenine dinucleotide (oxidized form) were compounded with the test substances below. A mixture was created consisting of about 200 mg nicotinamide adenine dinucleotide (oxidized form) and 10 ml of each test substance. Each ingredient was combined and mixed in a unified fashion with the NAD+ in separate beakers. Each mixture was placed in a sealed 15 ml beaker removed from UV light for one week at room temperature. After a one week period, the following ingredients were tested by performing an HPLC UV test.

Compounds that were found to be effective additives to nicotinamide adenine dinucleotide (oxidized form) preparations were ones that minimized the amount of degradation present and which did not cause rapid decomposition of nicotinamide adenine dinucleotide (oxidized form) as compared to a control which included only NAD+ in a gelatin capsule. These results are shown in Tables 3 and 4. Indeed, Table 3 indicates that the additives therein may have prevented the degradation of NAD+ better than merely placing the NAD+ in a gelatin capsule.

TABLE 3

| | | Sample | | | |
|---|---|---|---|---|---|
| | NAD+ | weight | Volume | Concentration | Recovery |
| Test substance | (mg) | (mg) | (ml) | (mg/ml) | (%) |
| MCT oil | 207.5 | 216.5 | 5 | 0.968 | 99.6 |
| Mineral oil | 200.4 | 330.8 | 6 | 1.323 | 99.1 |
| Vegetable oil | 204.5 | 234.7 | 5 | 1.08 | 98.6 |
| NAD+ in | | 9.6 | 10 | 0.928 | 96.7 |
| gelatin capsule | | | | | |
| NAD+ pure | 9.5 | 9.5 | 10 | 0.945 | 99.5 |

Table 3 shows that the NAD+, when added to a gelatin capsule, degraded. Without wishing to be bound by theory, this is believed to be due to the water content that is present in a gelatin capsule.

TABLE 4

| | | Sample | | | |
|---|---|---|---|---|---|
| | NAD+ | weight | Volume | Concentration | Recovery |
| Test substance | (mg) | (mg) | (ml) | (mg/ml) | (%) |
| 70% Sorbitol | 201.5 | 890.1 | 14 | 0.854 | 99.2 |
| Tocopherol | 202.2 | 440.8 | 14 | 0.67 | 99.6 |
| acetate | | | | | |
| Benzyl alcohol | 201.9 | 1201.2 | 20 | 1.162 | 99.6 |
| Polysorbate 80 | 205.8 | 125.2 | 14 | 0.216 | 99.1 |
| Glycerin | 201 | 195.2 | 10 | 0.385 | 99.5 |
| NAD+ pure | 8.5 | 8.5 | 10 | 0.849 | 99.9 |

As can be seen from Tables 3 and 4, the following test substances were found to be effective additives to nicotinamide adenine dinucleotide (oxidized form) preparations that did not degrade the NAD+:

Sorbitol (sugar alcohol)

Benzyl alcohol

Tocopherol acetates

Polysorbate 80

Glycerin

MCT oil

Mineral oil

Vegetable oil

Thus, the present inventor has found an answer to problems which were previously unknown, namely, how to modify the pH and physical properties of NAD+ preparations, without permitting the degradation of the NAD+. The addition of hygroscopic elements such as those discussed in the present disclosure, allows for the preparation of useful delivery agents for NAD+ for use in nutritional supplements, medical foods, and drugs.

### Example 5

PH testing was done by using both pH testing strips and a probe and meter for each test substance to determine its pH. Each test substance was dissolved in 150 ml distilled water then tested for its pH value. Where the test substance could not be tested because it was not water soluble, an art-recognized pH was used. Test substances are shown in Table 5 and include:

TABLE 5

| Test substance | (pH) Value |
|---|---|
| Potassium Hydroxide | 10.9 |
| KOH | |
| Magnesium Oxide MgO | 9.1 |
| Magnesium hydroxide MgOH₂ | 9.5 |
| Citric acid | 2.2 |
| Calcium Phosphate Dibasic | 8.5 |
| Stearic acid | 6.6 |
| Calcium Carbonate CaCO₂ | 9.4 |
| Magnesium stearate | Did not test - not water soluble |
| | pH range 7.0-9.0 |
| Corn starch | Did not test - not water soluble |
| | pH range 5.9-7.5 |
| Sodium Bicarbonate Na₂CO₂ | 9 |
| Potassium Bicarbonate | 9 |
| K₂CO₂ | |
| Mannitol | 6.7 |
| Magnesium Hydroxide | 12.8 |
| Mg(OH)₂ | |
| Dicalcium Phosphate Dihydrate | 6.8 |
| Sodium Hydroxide NaOH | 12.8 |
| Rice flour | Did not test - not water soluble |
| | (pH) range 6.0-6.8 |
| Lactose | 6.4 |
| Sodium Chloride NaCl | 6.9 |
| Dextrose | 6.4 |
| Ascorbyl palmitate | Did not test - not water soluble |
| | (pH) range 6.8-7.2 |
| Stevia | 6.2 |
| Erythitol | 6.3 |
| Sucrose | 6.8 |
| Xylitol | 6.5 |
| Ascorbic acid | 3.5 |
| Sodium ascorbate | 6.8 |
| Polyvinylpyrrolidone | 6.6 |
| Silicon dioxide | Did not test - not water soluble |
| | (pH) range 6.0-6.5 |

### Example 6

PH testing was done by using both ph testing strips and a probe and meter for each test substance to determine its pH. Each test substance was dissolved in 150 ml distilled water then tested for its pH value. Test substances are shown in Table 6 and include:

TABLE 6

| Test substance | (pH) Value |
|---|---|
| Vegetable oil | No pH |
| Mineral oil | No pH |
| MCT oil | No pH |
| Tocopherol acetate | Did not test to viscus |
| | (pH) neutral |
| Benzyl alcohol | 6.9 |
| Polysorbate 80 | Did not test to viscus |
| | (pH) neutral |
| Sorbitol | Did not test to viscus |
| | (pH) neutral |
| Glycerin | Did not test to viscus |
| | (pH) neutral |

Vegetable oil, mineral oil, and MCT oil have no water content and no pH value, so they appear to be ideal ingredients to formulate with NAD+. Tocopherol acetate, benzyl alcohol, polysorbate 80, sorbitol, and glycerin all have minimal water content and neutral pH values, also making them potentially beneficial ingredients to formulate with NAD+.

### Comparative Example 1

An example of a sublingual lozenge tablet according that showed extreme degradation after 4 months was a composition that included 80% mannitol (used as a binding agent and taste modifier), 5.66%, potassium bicarbonate (used as a pH buffer), 4% SOLUTAB (used as a binding agent), 3% stearic acid (used as a releasing agent), 1% green tea extract (used as a taste modifier), 1% erythritol (used as a taste modifier), 1% vanilla (used as a taste modifier), 1% stevia (used as a taste modifier), and 3.33% NAD+. The lozenge tablet was pressed into a tablet using a tablet mold and inserted into a sealed 75cc bottle with a 2 gram desiccant pouch. After 4 months the bottle was unsealed and the lozenge tablet was removed for testing. The lozenge tablet was not white in color, but instead had a greyish tint. The NAD+ was then tested using a HPLC UV test which resulted in a finding that the NAD+ had degraded to 65.8%. These results are shown in Table 1. Even though the NAD+ was stored in a desiccated state, and thus it would have been expected that the NAD+ would hold its stability, the NAD+ degraded. This illustrates that certain pH buffers, binding agents, taste modifiers, releasing agents, and bulking agents cause decomposition of the NAD+ molecule.

### Example 7

Based on the results of preceding examples, the present inventor found the following anhydrous ingredients to both have a pH greater than 4 and less than 9 and also be effective additives to NAD+ preparations to minimize degradation:

Calcium phosphate dibasic, CaHPO4.2H₂O (pH Modifier and buffer)

Stevia (taste modifier)

Xylitol (taste modifier)

Sucrose (taste modifier)

Erythritol (taste modifier)

Dextrose (taste modifier)

Rice flour (bulking agent)

Silicon dioxide (releasing agent)

Sodium chloride, NaCl (taste modifier)

Mannitol (taste modifier and lozenge binder)

Ascorbyl palmitate (releasing agent)

Microcrystalline cellulose (bulking agent)

Lactose (taste modifier and lozenge binder)

Magnesium carbonate (MgCO2) (pH modifier and buffer)

Sodium ascorbate (taste modifier)

Interestingly, the present inventor found that out of the ten pH buffers tested, only two (calcium phosphate dibasic and magnesium carbonate) had a pH of from 4-9 and did not degrade the NAD+. After comparing HPLC UV and pH test results, it can be seen that ingredients with pH values at or above approximately 9 and at or below approximately 4 generally showed significant decomposition of the NAD+.

Thus, the present inventor was able to provide a method for determining which materials may be suitable for use with NAD+ by relying on the pH of the material in addition to its hygroscopic tendencies. Under such a test, potassium bicarbonate, sodium bicarbonate, potassium hydroxide, sodium hydroxide, magnesium hydroxide, calcium carbonate, and magnesium oxide would not be deemed effective due to their high pH value of approximately 9 or above. Dicalcium phosphate dihydrate was one pH buffer that showed degradation from the HPLC UV test but had a fairly neutral pH. Although not wishing to be bound by theory, it is believed that this can be explained due to the fact that its molecular structure contains water.

Also, surprising was under this method, out of the eleven taste modifiers tested, two (citric acid and ascorbic acid) showed degradation in the HPLC UV test and were ruled out as acceptable ingredients based on their pH values being at or below a pH of 4, whereas under the prior art methods, all eleven would have been expected to have been successful. Sodium ascorbate, mannitol, lactose, sodium chloride, dextrose, stevia, erythritol, sucrose, and xylitol all showed positive results from the HPLC UV test which, without wishing to be bound by theory, may be explained by their fairly neutral pH levels.

Under this method, the binding agent results only showed polyvinylpyrrolidone as not being an acceptable ingredient to be used in formulations. Even though it has a fairly neutral pH value, it can absorb excess amounts of water and would be considered hygroscopic. Microcrystalline cellulose, dextrose, mannitol, lactose, and sucrose all tested highly in the HPLC UV test which, without wishing to be bound by theory, may be explained by their neutral pH values and their low hygroscopic tendencies.

Under this method, for the releasing agents, ascorbyl palmitate and silicon dioxide did not degrade the NAD+ and have neutral pH values, while stearic acid and magnesium stearate were found by the present inventor to cause degradation of the NAD+, perhaps due to their high pH alkaline values.

The bulking agents tested were corn starch and rice flour. Although they both have neutral pH levels, corn starch caused degradation of the NAD+ due to its hygroscopic properties, while rice flour did not.

Even though certain specific embodiments are thoroughly described in the present application, it should be understood that the same concepts disclosed with respect to those specific embodiments are also applicable to other embodiments, such as, for example, those embodiments relating to nasal, sublingual, vaginal, rectal and topical applications containing NAD+.

1-14. (canceled)

15. A composition comprising NAD+ and one or more components selected from the group consisting of hygroscopic pH modifiers, hygroscopic taste modifiers, hygroscopic binding agents, and hygroscopic release agents, wherein the pH of the hygroscopic pH modifiers, hygroscopic taste modifiers, hygroscopic binding agents, and hygroscopic release agents is not less than 4 and not greater than 9, and
wherein the composition does not comprise a hygroscopic pH modifier, hygroscopic taste modifier, hygroscopic binding agent, or hygroscopic release agent that has a pH less than 4 or greater than 9.

16. The composition according to claim 15, in which the one or more components are selected from the group consisting of magnesium carbonate, lactose, stevia, and silicon dioxide.

17-18. (canceled)

19. The composition according to claim 15, which does not contain any material having a pH less than 4 or greater than 9.

20. A composition comprising NAD+ and a hygroscopic compound which does not provide for the redox reaction of the NAD+ and which maintains the pH of the composition to be from 4-9, wherein said composition does not comprise a hygroscopic compound having a pH below 4 or above 9.

21. The composition according to claim 20, wherein the hygroscopic compound is a pH modifier that maintains the pH of the composition to be between 6.5 and 7.5.

22. The composition according to claim 20, wherein said pH modifier is selected from the group consisting of magnesium carbonate and calcium phosphate dibasic (CaHPO₄).

23. The composition according to claim 20, further comprising a taste modifier that does not provide for a redox reaction of the NAD+.

24. The composition according to claim 23, wherein the taste modifier is selected from the group consisting of mannitol, lactose, sodium chloride, dextrose, stevia, erythritol, sucrose, and xylitol.

25. The composition according to claim 20, further comprising a binding agent that does not provide for a redox reaction of the NAD+.

26. The composition according to claim 25, wherein the binding agent is selected from the group consisting of mannitol, microcrystalline cellulose, dextrose, and sucrose.

27. The composition according to claim 20, further comprising a releasing agent that does not provide for a redox reaction of the NAD+.

28. The composition according to claim 27, wherein the releasing agent is selected from the group consisting of ascorbyl palmitate, and silicon dioxide.

29. The composition according to claim 20, further comprising a coating that does not contain water.

30. The composition according to claim 20, wherein said composition is formed as a sublingual lozenge, tablet, or drop; nasal sprays or powder; topical lotion or cream; inhalation preparation; transdermal patch; or vaginal or rectal suppository.

31. The composition according to claim 20, wherein said composition is white.

32-34. (canceled)

35. A liquid composition comprising NAD+ and a liquid solvent that has a pH of greater than or equal to 4 and less than or equal to 9, wherein said liquid solvent does not provide for a redox reaction of the NAD+.

36. The liquid composition of claim 35, wherein said liquid composition contains less than 2% water by weight.

37. The liquid composition according to claim 35, wherein said liquid solvent is selected from the group consisting of oils, alcohols, acids, tocotrienols, and sugar alcohols.

38. The liquid composition according to claim 37, wherein said oils, alcohols, acids, tocotrienols, and sugar alcohols do not contain water in an amount that will cause a redox reaction of the NAD+.

39. The liquid composition according to claim 35, wherein said an aqueous solution and nonaqueous solvents is selected from the group consisting of MCT oil, mineral oil, vegetable oil, sorbitol, tocopherol acetate, benzyl alcohol, polysorbate 80, and glycerin.

40-41. (canceled)
